(19) United States
(12) Patent Application Publication
(10) Pub. No.: US 2024/0255530 A1
(43) Pub. Date: Aug. 1, 2024

(54) METHODS FOR DIAGNOSING AND DIFFERENTIATING SYNUCLEINOPATHIES

(71) Applicant: Qatar Foundation for Education, Science and Community Development, Doha (QA)

(72) Inventor: Omar El-Agnaf, Doha (QA)

(21) Appl. No.: 18/566,559

(22) PCT Filed: Jun. 2, 2022
(86) PCT No.: PCT/QA2022/050010

§ 371 Date: Dec. 1, 2023

Related U.S. Application Data
(60) Provisional application No. 63/195,903, filed on Jun. 2, 2021.

(51) Int. Cl. G01N 33/68

(52) U.S. Cl. CPC G01N 33/6896; G01N 2333/4728; G01N 2800/2821

(57) ABSTRACT

Provided are methods of diagnosis and differentiation of and between PD and DLB tissue samples.

### CROSS-REFERENCE TO RELATED APPLICATION

This application claims the benefit of, and priority to, U.S. Provisional Patent Application 63/195,903 filed Jun. 2, 2021, the entire contents of which are incorporated by reference herein.

### TECHNICAL FIELD

This disclosure relates to methods for diagnosis and differentiation of synucleinopathies—neurodegenerative disorders in which αSyn accumulates abnormally to form insoluble inclusions in the cell bodies or axons of neurons or oligodendrocytes.

### BACKGROUND

The synuclein protein was originally identified through several independent lines of investigation. In 1985, a neuron-specific protein of 143 amino acids (aa) was identified in Torpedo californica cholinergic synaptic vesicles. Later studies in amyloid plaques from an Alzheimer's disease (AD) brain discovered two unknown peptides, in addition to the major amyloid beta fragment, which were named NAC (non-A beta component of AD amyloid) peptide and its precursor, NACP and identified two proteins of 140 and 134 aa, which were highly expressed in the human brain. These results revealed the existence of a new protein family expressed predominantly in presynaptic nerve terminals. The 140 aa protein was named α-synuclein, while the 134 aa protein β-synuclein. The third and last protein of the family, γ-synuclein, was found to be highly expressed in ovarian and breast carcinomas.

Alpha-synuclein (αSyn) is primarily found in neural tissue, making up as much as one percent of all proteins in the cytosol of brain cells. It is predominantly expressed in the neocortex, hippocampus, substantia nigra, thalamus, and cerebellum, but can also be found in the non-neuronal glial cells. αSyn is encoded by the SNCA gene.

αSyn in solution is considered to be an intrinsically disordered protein, i.e. it lacks a single stable 3D structure. The normal conformation of α-synuclein is either a disordered monomer or an alpha-helical, multimeric conformation.

αSyn modulates DNA repair processes, including repair of double-strand breaks (DSBs). DNA damage response markers co-localize with alpha-synuclein to form discrete foci in human cells and mouse brain. Depletion of αSyn in human cells causes increased introduction of DNA DSBs after exposure to bleomycin and reduced ability to repair these DSBs. In addition, αSyn knockout mice display a higher level of DSBs, and this problem can be alleviated by transgenic reintroduction of human αSyn.

Synucleinopathies are a group of neurodegenerative disorders in which αSyn accumulates abnormally to form insoluble inclusions in the cell bodies or axons of neurons or oligodendrocytes. These disorders include Parkinson's Disease (PD), Dementia with Lewy bodies (DLB) and Multiple System Atrophy (MSA). The misfolding and aggregation of αSyn in neurons, neuronal processes, or glial cells is considered to be the underlying cause of synucleinopathies. The precise genetic and/or environmental trigger for αSyn misfolding still remains unknown; however, genetic mutations, mitochondrial dysfunction, proteolytic systems failure and neuroinflammation have been proposed to facilitate α-synuclein spread in the diseased brain.

In PD and DLB, αSyn inclusions are detected in Lewy bodies (LBs) and Lewy neurites (LNs) in subcortical and cortical neurons, while in MSA, αSyn inclusions are mainly detected in glial cells and are referred to as glial cytoplasmic inclusions (GCIs). The presence of αSyn aggregates in specific cell types provides the phenotypic differences observed between the different synucleinopathies.

These differences in presentation are potentially due to variable solubilities of αSyn species in specific synucleinopathies, and have been proposed to account for the existence of different αSyn strains able to spread from cell to cell in a prion-like manner. Moreover, different synucleinopathies show regional variation in the brain of both the initiation and progression of αSyn pathology, which could in turn affect the intrinsic structure of αSyn aggregates. However, methods of differentiating PD from DLB are lacking.

Therefore, improved diagnostic methods are of critical importance in this field.

### SUMMARY

Disclosed embodiments comprise methods for diagnosing and differentiating patients with synucleinopathies. For example, C-terminal truncations of monomeric αSyn (C-αSyn) species in PD and DLB have been shown to enhance the formation of amyloid aggregates, and can be important in disease progression and differentiating PD from DLB, thereby aiding diagnosis and treatment of these disorders.

Disclosed embodiments further comprise treatments of synucleinopathies.

### BRIEF DESCRIPTION OF THE DRAWINGS

FIGS. 1A and 1B shows characterization of αSyn monomeric substrates and real-time quaking-induced conversion (RT-QuIC) assay reaction buffer.
a. (FIG. 1A) The secondary structure of the three monomeric recombinant αSyn forms tested by circular dichroism (CD) show a minimum mean residue ellipticity at 195 nm, typical of disordered proteins with random coil;
b. (FIG. 1B) Effect of RT-QuIC reaction buffer on αSyn seeding activity; the RT-QuIC assay was performed in two different reaction mixtures (PIPES or PBS) for αSyn 1-140, αSyn 1-130 or αSyn 1-115 as substrates and the TBS fractions extracted from the Temporal Cortex samples of three PD patients as seeds.

FIG. 2 depicts RT-QuIC assays with recombinant αSyn forms in RT-QuIC using brain homogenates from PD and DLB cases. Aqueous-soluble and detergent-soluble (Cell Lytic, CL) fractions were prepared from the temporal and frontal cortices of 8 cases of DLB, PD or healthy controls (HC), then assayed by RT-QuIC using with three αSyn forms (full length 1-140 and truncated forms 1-130 or 1-115) as described in the Examples. Data shown are the averages (±SEM) of ThT signal (relative fluorescence units; RFU) over time.

FIG. 3 shows comparisons of the aggregation of αSyn forms in RT-QuIC using brain homogenates from PD and DLB cases. TBS-aqueous and detergent-soluble (Cell Lytic, CL) fractions were prepared from temporal (FIG. 3A) and frontal (FIG. 3B) cortices of 8 cases of DLB, PD or healthy controls (HC), then assayed by RT-QuIC using with three different αSyn forms (full length 1-140 and truncated forms 1-130 or 1-115). The RT-QuIC reactions are shown in FIG. 2. The box plots (median marked with a line and with maximum and minimum bars) summarize the area under the RT-QuIC curves (AUC). One-way ANOVA with Tukey's multiple comparison testing (GraphPad Prism) was used for statistical comparisons. Hashtags denote significant differences in comparison to healthy controls (HC); #P<0.05, ##P<0.01, ###P<0.001, ####P<0.0001. Asterisks denote significant differences within DLB or PD; *P<0.05, **P<0.01, ***P<0.001, ****P<0.0001.

FIG. 4 depicts PK digestion of RT-QuIC end products from the frontal cortex of PD and DLB cases. The RT-QuIC end products (as described in FIGS. 3 and 4) were collected and incubated at 37° C. with increasing concentrations of PK (0, 0.4, 4 μg/mL). The Figure shows two panels wherein each panel a representative immunoblot is shown for RT-QuIC PK digestion reactions, and a bar graph summarizing immunoblot quantification (mean±SD) from three cases for each condition. Immunoblots were quantified using ImageJ to calculate the intensity of aggregates (28 kDa) and PK-digested products (<14 kDa) relative to the intensity of the monomeric αSyn in each lane. Statistical analysis was done with GraphPad Prism using Two-way ANOVA with Tukey's post-test. Asterisks denote significant differences within a condition (*p<0.05, **p<0.01) and hashtags denote differences across groups (#p<0.05).

FIG. 5 shows PK digestion of RT-QuIC end products from the temporal cortex of PD and DLB cases. The RT-QuIC end products (as described in FIGS. 3 and 4) were collected and incubated at 37° C. with increasing concentrations of PK (0, 0.4, 4 μg/mL). The figure shows two panels where in each panel a representative immunoblot is shown for RT-QuIC PK digestion reactions, and a bar graph summarizing immunoblot quantification from three cases for each condition. Immunoblots were quantified using ImageJ to calculate the intensity of aggregates (28 kDa) and PK-digested products (<14 kDa) relative to the intensity of the monomeric αSyn in each lane. Statistical analysis was done with GraphPad Prism using Two-way ANOVA with Tukey's post-test. Asterisks denote significant differences within a condition (***p<0.001) and hashtags denote differences across groups (#p<0.05).

FIGS. 6A, 6B, and 6C show morphological characterization of C-terminally truncated RT-QuIC end products by TEM.
a. (FIG. 6A) Representative TEM images of RT-QuIC end products with the samples derived from PD and DLB temporal and frontal cortices;
b. (FIG. 6B) Area of aggregated fibril; and
c. (FIG. 6C) length of fibrils calculated from the TEM images of the RT-QuIC end products using ImageJ. Bar graphs show the average +SEM (n=3 of all samples, except for sample PD TBS Frontal 1-115, n=2).

One-way ANOVA with Tukey's multiple comparison testing (GraphPad Prism) was used for statistical comparisons; *P<0.05, **P<0.01. The dotted lines in panels B and C show the average fibril area and fibril length from the temporal cortex samples, respectively.

### DETAILED DESCRIPTION

Currently, diagnosis of PD and DLB is based on physical characteristics rather than molecular evidence. For example, when motor deficits (e.g., tremor, bradykinesia, rigidity) precede and are more severe than cognitive impairment, PD is usually diagnosed. When early cognitive impairment (particularly executive dysfunction) and behavioral disturbances predominate, DLB is usually diagnosed.

Disclosed embodiments comprise methods for diagnosing and differentiating patients with synucleinopathies. For example, C-terminal truncations of monomeric αSyn (C-αSyn) species in PD and DLB have been shown to enhance the formation of amyloid aggregates, and can be important in evaluating disease progression and differentiating PD from DLB, thereby aiding diagnosis and treatment of these disorders.

Aggregated αSyn protein is a core pathological feature of PD and DLB. One potential mechanism of how αSyn pathology spreads in PD and DLB is by template-directed aggregation of αSyn.

Both PD and DLB demonstrate the presence of different intracellular α-synuclein (αSyn) species, including C-terminally truncated αSyn (C-αSyn), although it is unknown how C-αSyn species contribute to disease progression.

Disclosed embodiments comprise use of recombinant C-αSyn, and PD and DLB brain lysates as seeds in real-time quaking-induced conversion (RT-QuIC) assay to determine how C-αSyn is involved in disease development and stratification. In embodiments, comparing the seeding activity of aqueous-soluble fractions to detergent-soluble fractions, and using αSyn 1-130 as substrate for the RT-QuIC assay, the temporal cortex seeds differentiated PD and DLB from healthy controls.

In embodiments, αSyn 1-130 seeded by the detergent soluble fractions from PD frontal cortex samples demonstrated greater seeding efficiency compared to DLB frontal cortex samples. In embodiments, Proteinase K resistant (PKres) fragments from the RT-QuIC end products using C-αSyn 1-130 or C-αSyn 1-115 were more obvious in the frontal cortex samples compared to the temporal cortex samples, potentially in line with the progression of αSyn pathology in the brain.

In embodiments, morphological examinations of RT-QuIC end products show differences in the size of the fibrils between C-αSyn 1-130 and C-αSyn 1-115, in agreement with the RT-QuIC results. These data suggest that C-αSyn species in PD and DLB may be important in disease progression and potentially differentiate PD from DLB.

Subjects suitable for the disclosed methods and treatments can comprise, for example, mammals, such as humans or animals.

Disclosed embodiments comprise methods for dignosing and differentiating patients with synucleinopathies.

Disclosed embodiments comprise treatments of synucleinopathies. For example, disclosed embodiments comprise treatment of PD, DLB, and MSA.

### Definitions

“A” and “an” are used herein to refer to one or to more than one (i.e., to at least one) of the grammatical object of the article. Byway of example, “an element” means one element or more than one element.

“Comprise,” “comprising,” “include,” “including,” “have,” and “having” are used in the inclusive, open sense, meaning that additional elements may be included. The terms “such as”, “e.g.”, as used herein are non-limiting and are for illustrative purposes only. “Including” and “including but not limited to” are used interchangeably.

“Effective,” “effective amount,” and “therapeutically effective amount” refer to that amount of a pharmaceutical composition thereof that produces a beneficial result after administration.

“In vitro” refers to an artificial environment and to processes or reactions that occur within an artificial environment. In vitro environments include, but are not limited to, test tubes and cell culture. The term “in vivo” refers to the natural environment (e.g., an animal or a cell) and to processes or reaction that occur within a natural environment.

“Or” as used herein should be understood to mean “and/or”, unless the context clearly indicates otherwise.

“Parenteral administration” and “administered parenterally” are art-recognized terms, and include modes of administration other than enteral and topical administration, such as injections, and include, without limitation, retro-orbital, intraocular, intravenous, intramuscular, intrapleural, intravascular, intrapericardial, intraarterial, intrathecal, intracapsular, intraorbital, intracardiac, intradermal, intraperitoneal, transtracheal, subcutaneous, subcuticular, intra-articular, subcapsular, subarachnoid, intraspinal and intrasternal injection and infusion.

“Patient,” “subject,” or “host” to be treated by the subject method can mean either a human or non-human animal, such as a mammal, a fish, a bird, a reptile, or an amphibian.

“Pharmaceutically acceptable” or “therapeutically acceptable” refers to a substance which does not interfere with the effectiveness or the biological activity of the active ingredients and which is not toxic to a patient.

“Pharmaceutically acceptable carrier” is art-recognized, and includes, for example, pharmaceutically acceptable materials, compositions or vehicles, such as a liquid or solid filler, diluent, excipient, solvent, or encapsulating material, involved in carrying or transporting any subject composition from one organ, or portion of the body, to another organ, or portion of the body. Each carrier must be “acceptable” in the sense of being compatible with the other ingredients of a subject composition and not injurious to the patient. In certain embodiments, a pharmaceutically acceptable carrier is non-pyrogenic. Exemplary materials which can serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, sunflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations.

“Pharmaceutical composition” refers to a formulation containing the therapeutically active agents described herein in a form suitable for administration to a subject. In embodiments, the pharmaceutical composition is in bulk or in unit dosage form. The quantity of active ingredient in a unit dose of composition is an effective amount and can be varied according to the particular treatment involved. One skilled in the art will appreciate that it is sometimes necessary to make routine variations to the dosage depending on the age and condition of the patient. The dosage will also depend on the route of administration. In a preferred embodiment, the active ingredients are mixed under sterile conditions with a pharmaceutically acceptable carrier, and with any preservatives, buffers, or propellants that are required.

“Treatment” or “treating” refers to any therapeutic intervention in a mammal, for example a human or animal such as a companion animal, including: (i) prevention, that is, causing the clinical symptoms not to develop, e.g., preventing infection or inflammation from occurring and/or developing to a harmful state; (ii) inhibition, that is, arresting the development of clinical symptoms, e.g., stopping an ongoing infection so that the infection is eliminated completely or to the degree that it is no longer harmful; and/or (iii) relief, that is, causing the regression of clinical symptoms, e.g., causing a relief of fever and/or inflammation caused by or associated with a microbial infection. Treatment can comprise multiple administrations of compositions disclosed herein.

“Reducing”, “suppressing” and “inhibiting” have their commonly understood meaning of lessening or decreasing.

### Methods of Diagnosis

### Tissue Preparation

Disclosed embodiments comprise methods of diagnosing synucleinopathies, for example using patient tissue samples. For example, disclosed methods comprise obtaining a tissue sample from a patient suspected of having a neurodegenerative disease, for example a neurodegenerative disease characterised by the abnormal accumulation of aggregates of alpha-synuclein protein in neurons, nerve fibres or glial cells.

In embodiments, a patient suspected of having a neurodegenerative disease characterised by the abnormal accumulation of aggregates of alpha-synuclein protein in neurons can display executive dysfunction, behavioral disturbances, or combinations thereof.

In embodiments, the tissue sample can comprise, for example, brain tissue, such as in the form of a lysate. In embodiments, the brain tissue can comprise tissue obtained from the temporal or frontal cortices of a patient suspected of having a neurodegenerative disease. In embodiments, the neurodegenerative disease can comprise, for example, DLB or PD.

Further embodiments comprise methods of differentiating DLB-positive from PD-positive samples. For example, disclosed methods comprise obtaining a tissue sample from a patient suspected of having a neurodegenerative disease characterised by the abnormal accumulation of aggregates of alpha-synuclein protein in neurons, nerve fibres or glial cells. In embodiments, the tissue sample can comprise brain tissue, for example a brain tissue lysate. In embodiments, the brain tissue lysate can comprise tissue obtained from the temporal or frontal cortices of a patient suspected of having DLB or PD.

Disclosed embodiments can further comprise homogenization of the obtained tissue in a buffer, for example an aqueous buffer, such as with a glass tissue homogenizer and protease and phosphatase inhibitors. The samples can then be centrifuged, with the collected supernatant representing the aqueous-soluble fraction. In embodiments, the aqueous buffer can comprise, for example, TBS.

Further embodiments comprise resuspending the pellet from the initial centrifugation in a detergent buffer. For example, in disclosed embodiments, CelLytic buffer (Sigma) can be used as a detergent buffer. Disclosed embodiments can further comprise homogenizing the resuspended pellet, for example on ice, and centrifuging again to produce a detergent-soluble fraction.

### Diagnostic Assays

In embodiments, following separation of the aqueous-soluble and detergent-soluble fractions, an assay can be performed to quantify the seeding activity of the various fractions of the various tissues. In embodiments, following separation of the aqueous-soluble and detergent-soluble fractions, an assay can be performed to quantify the rate of seeding activity of the various fractions of the various tissues.

For example, in an embodiment, an RT-QuIC assay can then be performed using purified αSyn, for example purified, recombinant, αSyn. In embodiments, the recombinant αSyn can comprise, for example, at least one of full-length αSyn or truncated αSyn. In embodiments, truncated αSyn variants can be used, for example αSyn 1-130, or αSyn 1-115.

The assay can further comprise use of a buffer. For example, a suitable reaction mixture can comprise, for example, 0.1 M PIPES (pH 6.9), 0.1 mg/mL αSyn and 10 μM ThT.

Reactions can be performed, for example, in triplicate in black 96-well plates with a clear bottom with, for example, 85 μL of the reaction mix loaded into each well together with, for example, 15 μL of 0.1 mg/ml TBS-soluble or detergent-soluble fractions. In embodiments, the plate is then sealed with a sealing film and incubated at, for example, 37° C. for 100 hours with intermittent cycles of, for example, 1 minute shaking periods (such as at 500 rpm, double orbital) and, for example, 15 min rest periods throughout the indicated incubation time.

In embodiments, the incubation time for the reaction can be at least 12 hours, at least 24 hours, at least 48 at least 72 hours, at least 96 hours, at least 120 hours, or more.

In embodiments, the incubation time for the reaction can be at most 12 hours, at most 24 hours, at most 48 at most 72 hours, at most 96 hours, at most 120 hours, or more.

In embodiments, the incubation time for the reaction can be 12 hours, 24 hours, 48 hours, 72 hours, 96 hours, 120 hours, or more.

In embodiments, the shaking period can be at least 15 seconds, at least 30 seconds, at least 60 seconds, at least 90 seconds, at least 120 seconds, at least 180 seconds, at least 240 seconds, or more.

In embodiments, the shaking period can be at most 15 seconds, at most 30 seconds, at most 60 seconds, at most 90 seconds, at most 120 seconds, at most 180 seconds, at most 240 seconds, or more.

In embodiments, the shaking period can be 15 seconds, 30 seconds, 60 seconds, 90 seconds, 120 seconds, 180 seconds, 240 seconds, or more.

In embodiments, the rest period can be at least 5 minutes, at least 10 minutes, at least 15 minutes, at least 20 minutes, at least 30 minutes, or more.

In embodiments, fluorescence measurements such as ThT fluorescence measurements (expressed as arbitrary relative fluorescence units (RFU)) can be obtained, for example with bottom reads, every 15 min using 450±10 nm (excitation) and 480±10 nm (emission) wave-lengths. The final fluorescence value is the mean fluorescence value taken at 100 hours.

In embodiments, reads can be performed every 5 minutes, every 10 minutes, every 15 minutes, every 20 minutes, every 30 minutes, every 40 minutes, or the like.

In embodiments, a positive assay signal can be defined as RFU more than, for example, 5 standard deviation units (RFU>5 SD) above the mean of initial fluorescence at 100 hours. In embodiments, the sample is considered positive if at least, for example, two of the replicates were positive, otherwise the sample is classified as negative.

### Methods of Treatment

Disclosed embodiments comprise methods of treatment of synucleinopathies. In embodiments, such methods can comprise treatments tailored toward the particular synucleinopathy diagnosed as described above. For example, disclosed embodiments comprise administration of, for example, immunotherapy neuroprotection, anti-inflammatories, mitochondrial agents, stem cells, neurotrophic factors, and the like.

Further embodiments can comprise administration of levodopa, zonisamide, valproic acid, quetiapine, pimavanserin, αSyn targeting agents including NPT200-11 and ambroxol, melatonin, clonazepam, and combinations thereof.

Disclosed embodiments can comprise treatments such as reduction of inflammation, restoration of netransmitter signaling, and the like.

Disclosed treatments can further comprise prevention or slowing of disease progression.

Disclosed treatments can comprise reduction of α-syn synthesis, propagation and accumulation.

### Example 1

In this study, we sought to determine whether αSyn C-terminal truncations behave differently in an RT-QuIC based assay and allow distinction between the different synucleinopathies in brain lysates derived from temporal and frontal cortices of DLB and PD patients.

We compared the kinetics of aggregation of two C-terminal truncated αSyn species (αSyn 1-130 and αSyn 1-115) to that of full-length αSyn (αSyn 1-140). We also analyzed the RT-QuIC end products by proteinase K digestion and electron microscopy in order to gain further insights on the structures of the aggregates that are involved in different synucleinopathies.

### Materials and Methods

### Expression and Purification of Recombinant αSyn Proteins

Recombinant human full-length αSyn (1-140) (FI-αSyn140) was expressed from the pRKI72 plasmid containing full-length cDNA from the human SNCA gene. Truncated recombinant αSyn 1-130 (C-αSyn130) and 1-115 (C-αSyn115) were expressed from pET3A plasmids containing the cDNA sequence as previously described. Briefly, all the recombinant αSyn forms were expressed in E. Coli BL21 (DE3) and purified using size-exclusion and Mono Q anion exchange chromatography. For αSyn 1-115 the Mono Q anion exchange elution buffers was prepared at pH 9 to account for increasing isoelectric point in this truncated protein.

All recombinant proteins were diluted in 20 mM Tris/HCl pH 7.4, 100 mM NaCl, and protein concentrations were determined using the bicinchoninic acid assay (Pierce). Aliquots (300 μL of 1 mg/mL) were prepared and stored at −80° C. Prior to use, the proteins were filtered (100-kDa spin filter) and the protein concentration was again determined by bicinchoninic acid assay.

Isolation of TBS (Aqueous)-Soluble and Detergent-Soluble Fractions from Brain Tissue

Brain tissues from PD or DLB cases from the temporal or frontal cortex were prepared. Samples were homogenized with a glass tissue homogenizer at 10% (w/v) on ice in TBS (20 mM Tris-HCl pH 7.4, 150 mM NaCl) and 5 mM EDTA with protease and phosphatase inhibitors (Thermo Fisher Scientific). Samples were centrifuged at 3000×g, at 4° C. for 30 min. The collected supernatant represents the TBS (aqueous)-soluble fraction.

The pellet was then resuspended in CelLytic buffer (Sigma), homogenized as before on ice and centrifuged at 3000×g at 4° C. for 30 min. The resulting supernatant represented the detergent-soluble fraction.

The total protein concentration was measured in both fractions by BCA assay (Pierce, Thermo Fisher Scientific) and 0.1 mg/mL aliquots were prepared and stored at −80° C.

### Circular Dichroism Spectra

Circular Dichroism (CD) spectra were measured with Chirascan CD Spectrophotometer (Applied Photophysics) using a quartz cell with a 1-mm path length. All measurements were carried out using 5 μM αSyn in PBS pH 7.4. The CD spectra were obtained averaging five scans in the wavelength range of 195 nm-250 nm.

### RT-QuIC Assay

The RT-QuIC assay was performed using purified recombinant αSyn and re-optimized from a previously described method. The reaction buffer was composed of 0.1 M PIPES (pH 6.9), 0.1 mg/mL αSyn, and 10 μM ThT. Reactions were performed in triplicate in black 96-well plates with a clear bottom (Nunc, Thermo Fischer) with 85 μL of the reaction mix loaded into each well together with 15 μL of 0.1 mg/ml TBS-soluble or detergent-soluble fractions.

The plate was then sealed with a sealing film (Thermo Fisher Scientific) and incubated in a BMG Labtech FLUOstar OMEGA plate reader at 37° C. for 100 hours with intermittent cycles of 1 min shaking (500 rpm, double orbital) and 15 min rest throughout the indicated incubation time. ThT fluorescence measurements, expressed as arbitrary relative fluorescence units (RFU), were taken with bottom reads every 15 min using 450±10 nm (excitation) and 480±10 nm (emission) wave-lengths. The final fluorescence value was the mean fluorescence value taken at 100 hours.

A positive signal was defined as RFU more than 5 standard deviation units (RFU>5 SD) above the mean of initial fluorescence at 100 hours. The sample was considered positive if at least two of the replicates were positive, otherwise the sample was classified as negative.

### Proteinase K Digestion and Western Blotting

Proteinase K digestion of the reaction end products was performed using two different proteinase K concentrations (0.4 and 4 μg/ml) at 37° C. for 30 minutes. The reactions were stopped by adding NuPAGE LDS buffer and incubating the samples at 60° C. for 10 minutes.

Samples were analyzed by electrophoresis in 4%-12% Bis-Tris gels (Invitrogen) using MES as running buffer and immunoblotted on nitrocellulose membranes (Amersham). Blots were blocked in PBS containing 0.05% (v/v) Tween 20 (PBST) and 5% (w/v) non-fat dried skimmed milk powder and probed with Syn1 antibody (aa91-99 of human αSyn) at 50 ng/ml final antibody concentration in PBST. Blots were developed using ECL detection western blotting reagents (Pierce).

### Transmission Electron Microscopy (TEM)

Samples of αSyn RT-QuIC end products were transferred in a 5 μL volume to a TEM grid (S162 Formvar/Carbon, 200 Mesh, agar scientific). After 1 min, samples were fixed using 5 μL of 0.5% glutaraldehyde followed by one wash with 50 μL of ddH₂O. Five μL of 2% uranyl acetate were added to the grid, followed by placement of the grid in a sample holder.

### Statistical Analysis

All statistical analyses were performed using GraphPad Prism Software (Version 9.0.2, GraphPad Software, Inc., San Diego, CA). The types of tests performed are detailed in the Figure legends. P-values<0.05 were considered statistically significant.

### Results

### Characterization of αSyn Forms Used as Substrates and Effect of Buffer Composition on the RT-QuIC Assay

As the RT-QuIC assay is dependent on the quality of the substrate used in the reaction, we analyzed the recombinant αSyn monomers used in this study for their secondary structure by circular dichroism (CD). The CD spectra of the αSyn monomers showed negative bands at 195 nm, consistent with a random coil structure (FIG. 1A), and the molar ellipticity increased with increasing truncation of the C-terminus of the recombinant protein; FI-αSyn40 versus C-αSyn130 versus C-αSyn115. To optimize and implement the conditions for αSyn RT-QuIC, we tested two different reaction buffers, 100 mM piperazine-N,N′-bis(ethanesulfonic acid) (PIPES; pH 6.9) and 1× PBS (10 mM sodium phosphate, 138 mM NaCl, and 2.7 mM KCl; pH 7.4). As shown in FIG. 1B, only 100 mM PIPES (pH 6.9) showed an increase in the ThT fluorescence curves in the RT-QuIC assays with three different cases of PD; which began to increase at ˜15, 24 and 40 hours post-reaction with C-αSyn115, C-αSyn130 and FI-αSyn140, respectively.

Seeding Different Forms of αSyn in RT-QuIC Using Brain Homogenates from PD and DLB Cases

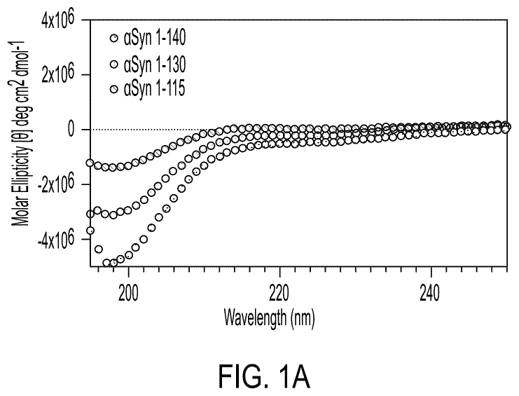

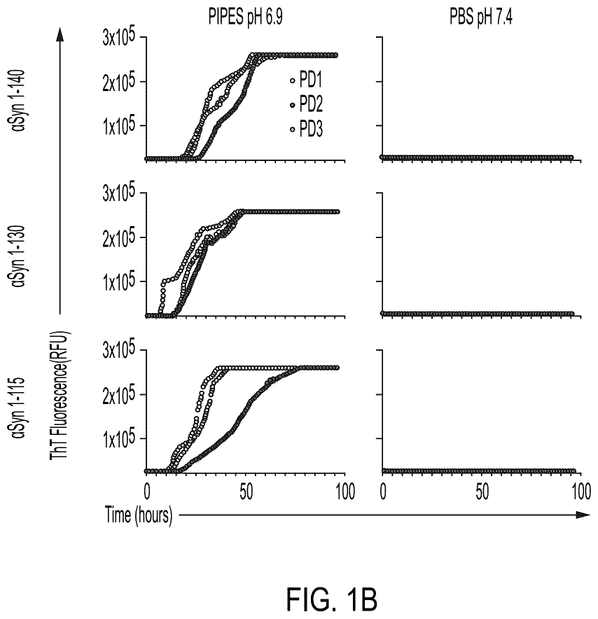

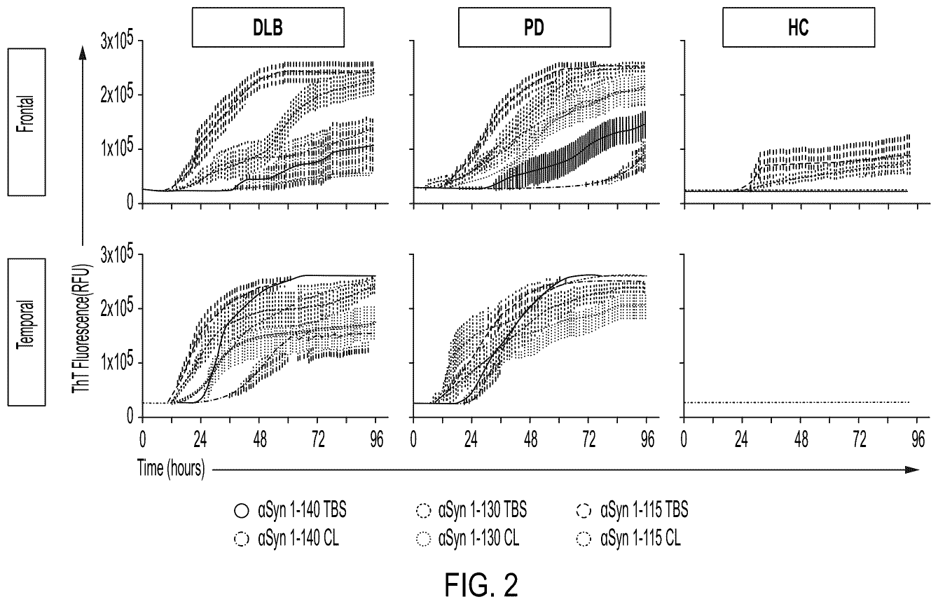

PIPES pH 6.9 was selected as the optimal working buffer and used to compare the three forms of αSyn in RT-QuIC assays. The average curves from the RT-QuIC assays are shown in FIG. 2 for reactions carried out with TBS-soluble or detergent-soluble fractions from the temporal or frontal cortices from PD or DLB cases against healthy controls (HC). These two different fractions were chosen to take into account the variable degree of solubility of the different amyloidogenic species present in brain samples.

There was a trend for faster reaction kinetics with increasing C-terminal truncation of αSyn, particularly for reactions seeded with extracts from the frontal cortex samples where the longest lag-phases were observed with FI-αSyn140 with aqueous soluble fractions (45±21 h for PD, 59±18 h for DLB, p>0.05) and detergent soluble fractions (54±6.4 h for PD). The shortest lag-phases in the frontal cortex were seen with C-αSyn115 with aqueous soluble fractions (20±5.4 h for PD, 20±6 h for DLB) and detergent soluble fractions (38±30 h for PD, 54.5±29 h for DLB). The lag-phase was significantly shorter with C-αSyn115 than FI-αSyn140 in the reactions seeded with the aqueous soluble and detergent soluble fractions extracted from the frontal cortex samples (p<0.05 and p<0.01, respectively).

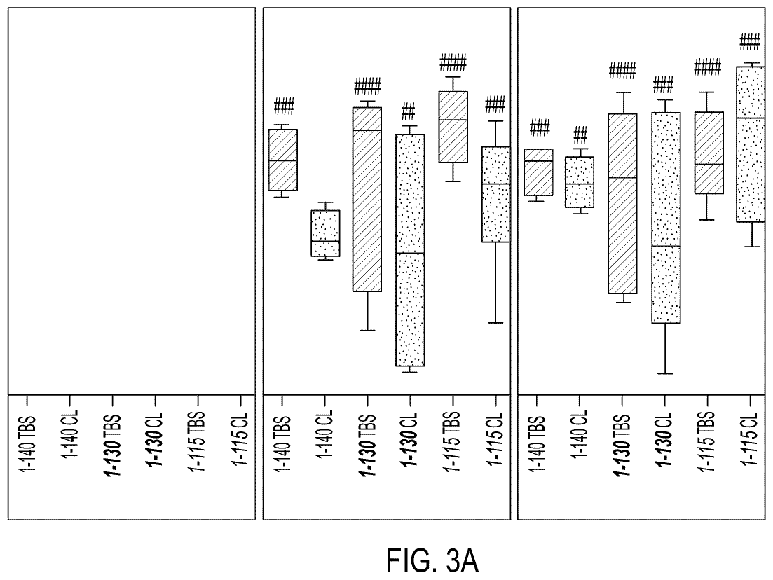

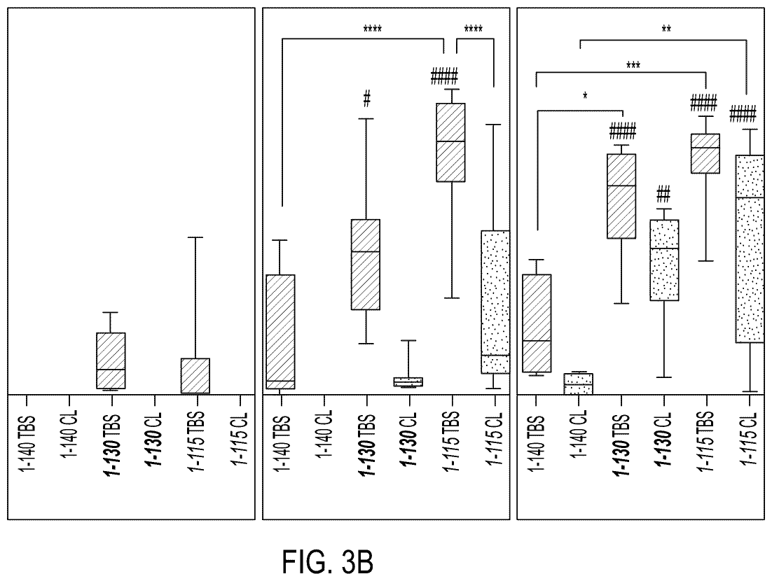

For a quantitative assessment, we defined the area under the RT-QuIC curves (AUC) as the seeding parameter of interest as it summarizes all the kinetic features of the aggregation, including the speed and extent of aggregation. The temporal cortex from DLB and PD showed significantly higher AUC for the three forms of αSyn used compared to that from healthy controls (p<0.05) and without significant differences between αSyn forms or between the TBS-soluble and detergent-soluble fractions (FIG. 3A). On the other hand, the frontal cortex from DLB and PD showed more variations (FIG. 3B). The C-αSyn forms showed faster aggregation with the TBS-soluble fractions of the frontal cortex from DLB and PD compared to the FI-αSyn140 and showed significantly faster aggregation than in controls (P<0.05). In the detergent-soluble fractions of DLB and PD, C-αSyn forms showed higher AUC than the FI-αSyn but only reached significance in PD cases (P<0.05). Aggregation of αSyn was faster in the TBS-soluble fractions than detergent-soluble fractions in the frontal cortex and reached significance using C-αSyn115 in DLB (P<0.0001). In the TBS-soluble fraction from DLB frontal cortex, aggregation was significantly faster than FI-αSyn140 (P<0.0001). In PD samples, both C-αSyn130 and C-αSyn115 had significantly faster aggregation rate than FI-αSyn140 when incubated with the TBS-soluble fractions (marked by asterisks in FIG. 3B), whereas only C-αSyn115 in detergent-soluble fractions from PD frontal cortex had significantly faster aggregation compared to αSyn 1-140 (**P<0.01).

The RT-QuIC data suggested that in the temporal cortex (FIG. 3A), both TBS- and detergent-soluble fractions from PD and DLB had similar seeding activity and promoted aggregation of different αSyn forms with minimal differences. Consequently, receiver operating characteristic (ROC) curve analyses showed that full-length and C-αSyn forms clearly differentiated PD or DLB from healthy controls using both TBS- and detergent-soluble fractions with 100% sensitivity and specificity (p=0.02 for αSyn 1-140 and p=0.007 for αSyn 1-130 and αSyn 1-115, Table 1A). However, none of the αSyn forms could differentiate PD from DLB using the temporal cortex. Whereas, in the frontal cortex (FIG. 3B), particularly using C-αSyn forms, the TBS-soluble fractions had greater seeding activity than the detergent-soluble fractions, and PD cases had greater seeding activity in the detergent-soluble fractions than in DLB cases. This was further illustrated by the ROC curve analysis of the frontal cortex data which showed that the detergent-soluble fraction, particularly with αSyn 1-130 could differentiate PD cases from DLB cases with 88% sensitivity and 100% specificity (p=0.001, Table 1B, red font).

TABLE 1
BOC curve analysis of RT-QulC data with PD and DLB cases with different [symbol] Syn forms.

| | [symbol] | | | | | | | |
|---|---|---|---|---|---|---|---|---|
| | TBS-Soluable Fraction | | | | Detergent-Soluable Fraction | | | |
| | | | | Cutoff | | | | Cutoff |
| Temporal | Sens. | Spec. | p * | (RT-QulC AUC) | Sens. | Spec. | p * | (RT-QulC AUC) |
| | [symbol] Syn 1-140 | | | | | | | |
| C [symbol] vs. HC | 100% | 100% | 0.00 [symbol] | > [symbol] ,027 | 100% | 100% | 0.007 | >4 [symbol] ,831 |
| PD vs. HC | 100% | 100% | 0.02 | > [symbol] ,027 | 100% | 100% | 0.02 | >5,343,731 |
| DLB vs. HC | 100% | 100% | 0.02 | > [symbol] | 100% | 100% | 0.02 | >4,010,801 |
| PD vs. DLB | 75% | 50% | 0.563 | < [symbol] ,992 | 100% | 7 [symbol] % | 0.0 [symbol] | >10,077, [symbol] |
| | [symbol] Syn 1-130 | | | | | | | |
| C [symbol] vs. HC | 100% | 100% | 0.00 [symbol] | > [symbol] ,147 | 94% | 100% | 0.003 | >1,3 [symbol] |
| PD vs. HC | 100% | 100% | 0.007 | > [symbol] ,490 | 100% | 100% | 0.007 | > [symbol] |
| DLB vs. HC | 100% | 100% | 0.007 | > [symbol] ,147 | 88% | 100% | 0.007 | >1, [symbol] |
| PD vs. DLB | 75% | 50% | 0. [symbol] | < [symbol] ,128 | 88% | 50% | 0. [symbol] 01 | >3,1 [symbol] |
| | αα [symbol] Syn 1-115 | | | | | | | |
| C [symbol] vs. HC | 100% | 100% | 0.003 | > [symbol] ,217 | 94% | 100% | 0.003 | >6,5 [symbol] |
| PD vs. HC | 100% | 100% | 0.007 | > [symbol] ,217 | 100% | 100% | 0.007 | >4, [symbol] |
| DLB vs. HC | 100% | 100% | 0.007 | > [symbol] 27 | 100% | 100% | 0.007 | >2,164,070 |
| PD vs. DLB | 63% | 75% | 0.142 | < [symbol] | [symbol] | [symbol] % | 0.20 [symbol] | <13, [symbol] ,774 |

| | b | | | | | | | |
|---|---|---|---|---|---|---|---|---|
| | TBS-Soluable Fraction | | | | Detergent-Soluable Fraction | | | |
| | | | | Cutoff | | | | Cutoff |
| Frontal | Sens. | Spec. | p * | (RT-QulC AUC) | Sens. | Spec. | p * | (RT-QulC AUC) |
| | [symbol] Syn 1-140 | | | | | | | |
| C [symbol] vs. HC | 100% | 100% | 0.006 | < [symbol] | 100% | 100% | 0.0 [symbol] | >64.66 |
| PD vs. HC | 100% | 100% | 0.02 | [symbol] ,271 | 100% | 100% | 0.02 | >72 [symbol] 9 |
| DLB vs. HC | 100% | 100% | 0.02 | > [symbol] ,870 | 100% | 100% | 0.00 [symbol] | > [symbol] 66 |
| PD vs. DLB | 75% | 100% | 0.2 [symbol] | <893,180 | 100% | 75% | 0.1 [symbol] | <200 [symbol] 163 |
| | [symbol] Syn 1-130 | | | | | | | |
| C [symbol] vs. HC | 100% | 100% | 0.001 | > [symbol] 17 | 100% | [symbol] % | 0.02 [symbol] | 0.7891 |
| PD vs. HC | 100% | 100% | 0.001 | > [symbol] 17 | 88% | 88% | 0.00 [symbol] | 0. [symbol] 37 [symbol] |
| DLB vs. HC | 88% | 88% | 0.00 [symbol] | > [symbol] ,272 | 88% | [symbol] % | 0.348 | [symbol] 18, [symbol] |
| PD vs. DLB | 75% | 88% | 0.093 | >10, [symbol] 42, [symbol] | 88% | 100% | 0.001 | [symbol] |
| | [symbol] Syn 1-115 | | | | | | | |
| C [symbol] vs. HC | 88% | 75% | 0.0 [symbol] | >11,421, [symbol] | 87% | 88% | 0.001 | > [symbol] ,687 |
| PD vs. HC | 7 [symbol] % | 88% | 0.0 [symbol] | >13, [symbol] ,264 | 88% | 88% | 0.002 | > [symbol] 1, [symbol] |
| DLB vs. HC | 88% | 75% | 0.16 | >11,421, [symbol] | 88% | 88% | 0.00 [symbol] | > [symbol] ,66 [symbol] |
| PD vs. DLB | 88% | [symbol] % | 0. [symbol] | <1 [symbol] ,073 | 75% | 75% | 0.248 | [symbol] ,097 |

* Statistical differences for the AUC comp [symbol] 0.5 using C [symbol] statistic were performed using GraphPad [symbol] . [symbol]
indicates data missing or illegible when filed

### Detection of Proteinase K (PK)-Resistant Fragments of C-Terminal Truncated α-Syn in the RT-QuIC End Products

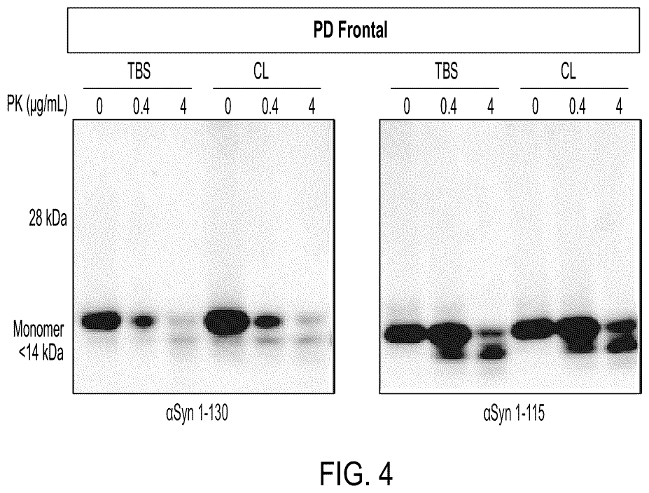

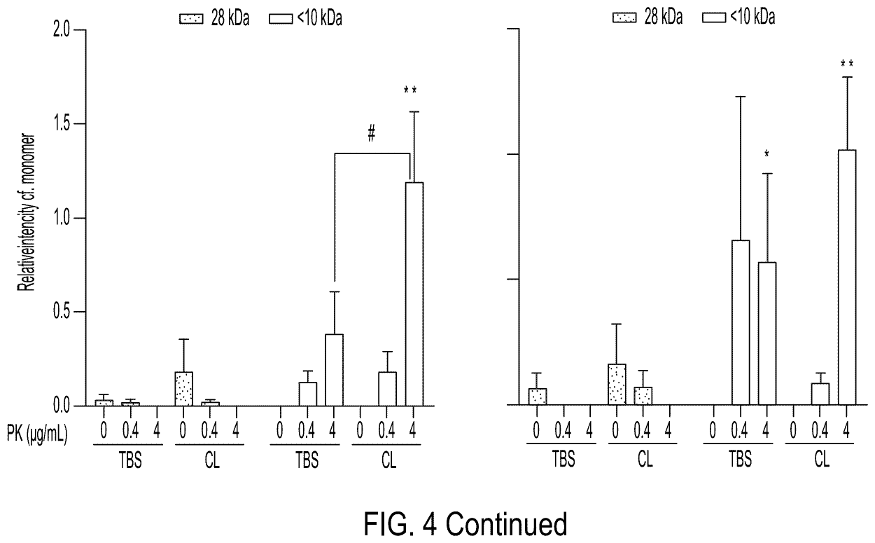

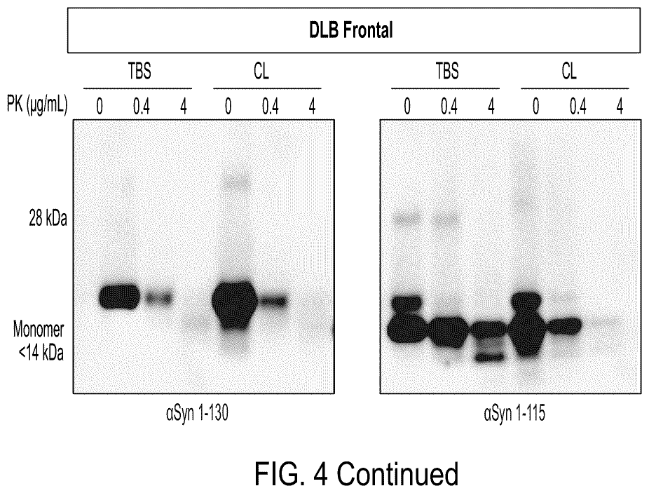

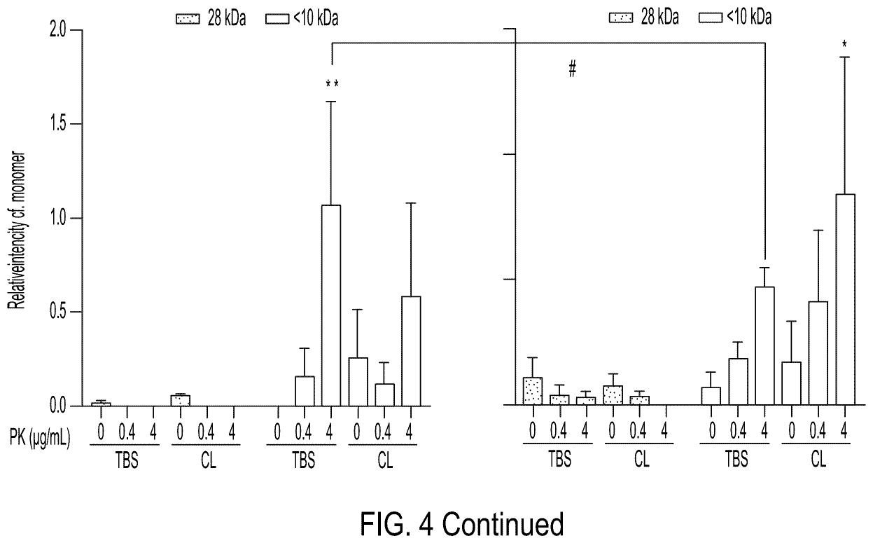

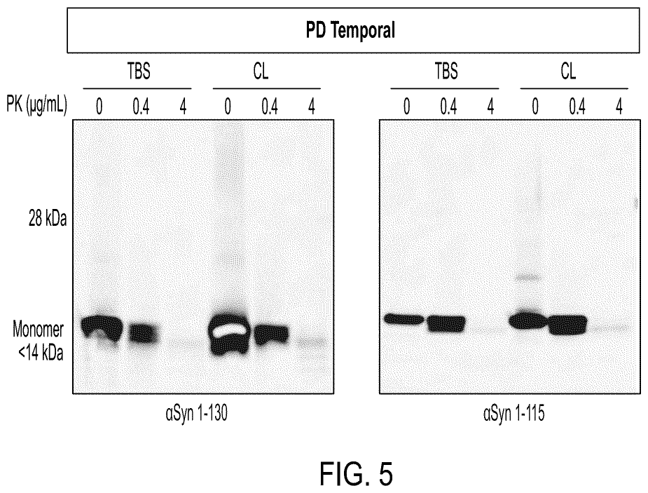

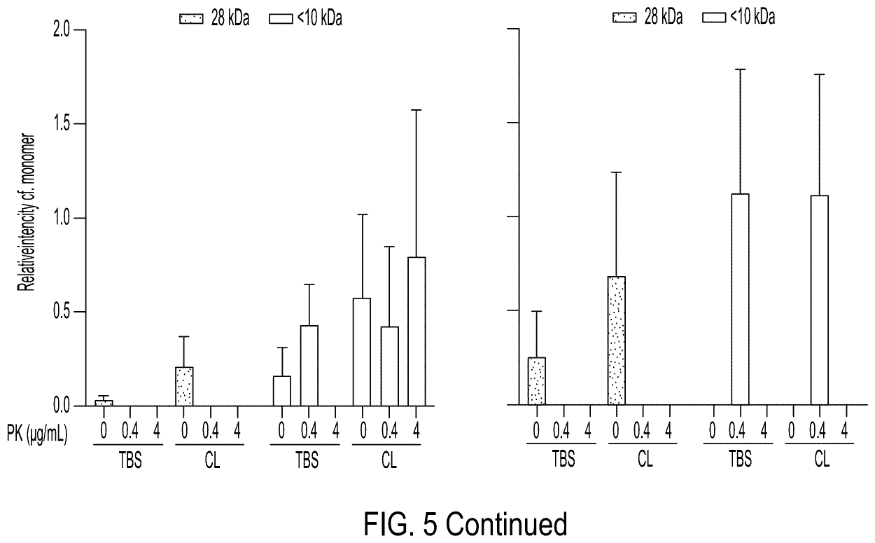

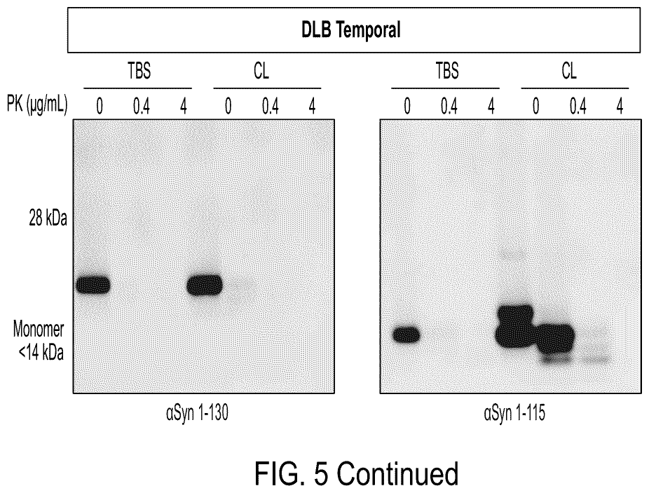

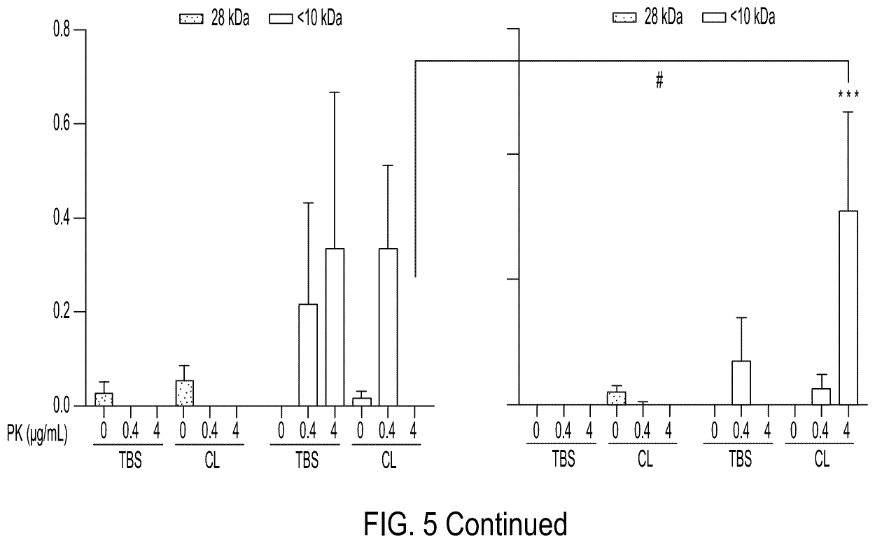

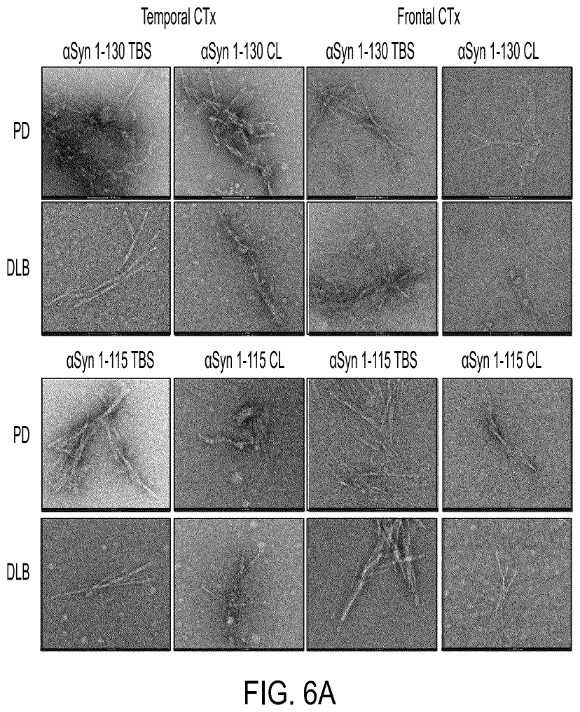

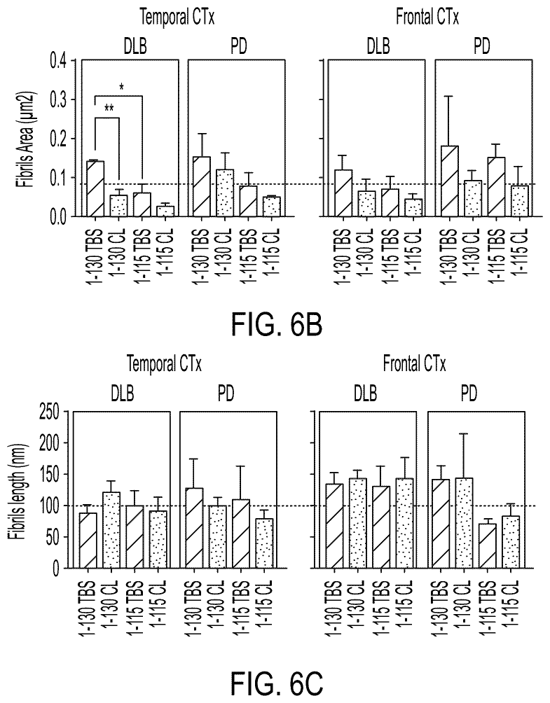

The end products from RT-QuIC assays using αSyn 1-130 and 1-115 forms were characterized by proteinase K (PK) digestion assay. Representative blots of PK digestion of the end products from RT-QuIC assays with PD and DLB are shown for frontal cortex samples (FIG. 4), and for temporal cortex samples (FIG. 5). Using either C-αSyn forms, dimers (28 kDa) were detected by immunoblotting before PK digestion, and were not significantly different across PD and DLB brain homogenates (n=3, FIG. 4 & FIG. 5, p>0.05 Two-way ANOVA).

PK^{res} fragments from αSyn 1-115 reactions were significantly higher with detergent-soluble fractions from PD and DLB frontal cortex, and only with aqueous soluble fraction from PD, compared to no PK digestion (FIG. 4). For the temporal cortex, PK^{res} fragments from αSyn 1-115 were significantly different only with the detergent-soluble fraction from DLB cases (FIG. 5). There were no significant differences between PD and DLB in PK^{res} fragments using αSyn 1-115.

Overall, for αSyn 1-130, PK^{res} bands were more obvious, although not statistically different (p>0.05), in the frontal cortex versus the temporal cortex, which may be in line with the progression of αSyn pathology in the brain. PK^{res} fragments were significantly higher in the detergent-soluble fractions compared to the aqueous-soluble fractions from PD frontal cortex (FIG. 4). When comparing PD to DLB with αSyn 1-130, PK^{res} bands (<14 kDa) in the TBS-soluble fractions were significantly higher in DLB versus PD (p<0.05, FIG. 4). The detergent-soluble fraction from PD frontal with αSyn 1-130 showed higher, but not significant (p>0.05), PK^{res} bands compared to DLB cases.

### Morphological Characterization of RT-QuIC End Products by TEM

The morphology of the RT-QuIC end products with truncated αSyn forms was analyzed by TEM. Representative images from single cases of PD and DLB are shown in FIG. 6A. To quantify any observed morphological differences, we compared the average fibril length and the area of the entire aggregate (FIG. 6B-C). TEM analysis did not show major differences between fibrils seeded with DLB or PD extracts. Overall, fibrils seeded with the detergent soluble fractions appeared smaller in size than those seeded with the aqueous soluble fraction, although the observed differences did not reach statistical significance except for DLB temporal samples with αSyn 1-130 (p<0.01, FIG. 6B). The fibrils in the frontal cortex appeared longer than the temporal cortex, particularly with αSyn 1-130, but the differences were not statistically significant (FIG. 6C).

### Discussion

There is currently increasing interest in the potential role of distinct species of αSyn contributing to the diverse clinical features and clinical phenotypes of synucleinopathies, but relatively little is known about whether such species differ in terms of their expression and abundance across different brain regions and between distinct forms of synucleinopathies. Therefore, the present study sought to investigate whether different brain regions in PD and DLB could be distinguished in terms of their seeding capacity. We tested whether employing C-terminal truncated αSyn, which has been demonstrated to be more aggregation prone than full-length αSyn, may enable distinction of different brain regions from one another in synucleinopathies, or differences between synucleinopathies themselves, using the RT-QuIC assay. Here we report that although temporal cortex tissue differentiated cases from controls in RT-QuIC, no differences in the seed-propensity of αSyn were observed across tissue fractions or between PD and DLB. In contrast, frontal cortex tissue did reveal differences in αSyn seeding, which was particularly efficient in distinguishing PD from DLB using detergent-soluble tissue fractions. We suggest these results indicate regional and phenotype-specific differences in αSyn-seeding propensity, which may relate to regionally diverse strains of αSyn, phenotype-associated αSyn pathology, or the maturity of aggregates between PD and DLB cortex.

Since the first report of aggregation assays such as real-time quaking induced conversion (RT-QuIC) for αSyn in both brain and CSF samples from patients affected by synucleinopathies, the RT-QuIC assay has been applied to a variety of biological samples such as brain tissue sample, CSF, the olfactory mucosa and the submandibular glands. Despite these successful results, the ability of RT-QuIC in stratifying synucleinopathies is still controversial, with recent studies reporting conflicting results. As standardization and harmonization of the αSyn RT-QuIC protocol between laboratories is lacking, it is possible that these different results are due to different experimental settings. For example, the use of different reaction buffers with different pH, the use of glass/silica beads, different shaking and rest times, and the purification method of the recombinant αSyn substrate, which can all influence the seeding kinetics and the robustness of αSyn RT-QuIC assay.

Moreover, different brain regions might also be relevant. PD and DLB pathologies are both characterized by αSyn accumulation in cortical and subcortical regions, with DLB patients showing higher αSyn expression levels in the superior temporal cortex than PD and healthy control cases. A small number of published studies comparing PD and DLB using RT-QuIC have found a higher seeding activity in DLB cases compared to PD cases when frontal cortex homogenates were used. This may be explained by the fact that deposition of αSyn aggregates in the frontal cortex is a prominent feature of DLB pathology while in PD cases without dementia the deposition is prominent is substantia nigra pars-compacta.

In the present study we used the RT-QuIC assay to compare αSyn aggregation propensity between PD and DLB against healthy controls in two different brain regions, the frontal and temporal cortex, using aqueous (TBS)- and detergent-soluble fractions. The αSyn aggregation propensity in these brain fraction as seeds was evaluated using three different αSyn forms as substrates, FI-αSy140 and C-terminally truncated (C-αSyn130 and C-αSyn115). To our knowledge, this is the first study to address αSyn aggregation propensity using different seeds and substrates as a possible approach to differentiate between different αSyn strains.

We found that RT-QuIC assay was unsuccessful using PBS (pH 7.4) as a reaction buffer, whereas PIPES (pH 6.9) was a more successful and reproducible conversion buffer. Remarkably, different fractions from different brain regions of PD and DLB cases showed different aggregation propensities, which was highlighted using different αSyn forms as substrates. It should be noted that previous studies have not evaluated RT-QuIC using the temporal cortex of synucleinopathies. In our study, RT-QuIC with the temporal cortex differentiated these synucleinopathies from healthy controls, but temporal cortex from PD and DLB had a similar aggregation propensity irrespective of the αSyn substrates or the nature of the fractions used, aqueous- or detergent-soluble. Consequently, RT-QuIC with the temporal cortex could differentiate PD and DLB cases from healthy controls with 100% sensitivity and specificity regardless to the fraction or recombinant αSyn substrate used.

In contrast, the frontal cortex of both PD and DLB cases showed higher aggregation propensity in the aqueous- versus detergent-soluble fractions. In line with this finding, a previous study using ELISA showed that the levels of αSyn oligomers in the aqueous-soluble fraction are elevated in the frontal cortex of PD and DLB cases (700-750 pg/mL) compared to the detergent-soluble αSyn oligomers (250-300 pg/mL). This ELISA-based study did not report differences in the levels of aqueous- or detergent-soluble αSyn oligomers from the frontal cortex between PD and DLB cases. This was corroborated in our study using RT-QuIC, however, another study using RT-QuIC reported a higher seeding activity in the frontal cortex of DLB cases compared to frontal cortex of PD cases. This discrepancy may be explained by differences in the brain homogenate preparation. Candelise et al. subjected the total frontal cortex homogenates to a complex, centrifugation-based purification method to isolate >100 kDa αSyn seeds from the samples, while our previous study and in this study direct extraction methods were used.

In the frontal cortex, we found that the aggregation propensity increased with increasing C-terminal truncation of αSyn substrates. Interestingly, while αSyn 1-140 differentiated PD and DLB from healthy controls with 100% sensitivity and specificity, the C-αSyn forms also demonstrated differences between PD and DLB. Remarkably, αSyn 1-130 as substrate with detergent-soluble fractions from the frontal cortex differentiated PD from DLB with 88% sensitivity and 100% specificity. Altogether, our data show that RT-QuIC using αSyn 1-130 substrate with the temporal cortex could differentiate PD and DLB from healthy controls, and it can differentiate PD from DLB using the detergent-soluble fraction from the frontal cortex. In contrast, αSyn 1-115 as substrate is not able to distinguish PD from DLB in both brain regions. However, αSyn 1-115 differentiated PD and DLB from healthy controls with 100% sensitivity and 100% specificity with the aqueous soluble fractions, and with 93% sensitivity and 100% specificity with the detergent soluble fractions from the temporal cortex.

One might speculate that the presence of αSyn strains with different cellular tropism might have the ability to transmit their conformational properties to the recombinant amyloid fibrils in RT-QuIC. In agreement with this observation, Shahnawaz et al. showed that the conformational properties of αSyn aggregates associated with PD and MSA can be transmitted by αSyn-PMCA. In this study, end products from RT-QuIC reactions using the C-terminally truncated αSyn forms showed more obvious PKres fragments in the frontal cortex than in the temporal cortex, suggesting the existence of αSyn strains with different biochemical properties. TEM analysis in this study suggested that reaction end products seeded with the aqueous-soluble fractions were larger in size than those form reactions seeded with the detergent-soluble fractions, which agrees with the increased seeding response observed in RT-QuIC compared to full length αSyn. Moreover, the fibrils detected by TEM of the RT-QuIC end products appeared longer in the frontal cortex than the temporal cortex. Taken together, these findings strongly suggest that αSyn aggregates show substantial heterogeneity even within individuals, with important implications for understanding the pathobiology of αSynucleinopathies, the development of therapeutics targeting αSyn, and personalized medicine.

Several studies have identified different C-terminal truncated forms of αSyn in αSynucleinopathies, and different aggregation properties of truncated forms have been studied in vitro. While αSyn forms have been quantified in brains of PD patients, to the best our knowledge, there are limited studies that compared the presence of C-terminal truncated αSyn forms in different αSynucleinopathies and in different brain regions. Interestingly, it has been reported that truncated αSyn is present in 70% of Lewy bodies and neurites in DLB versus 90% in PD. Moreover, another study has shown different levels of C-terminal truncated αSyn forms in diffused Lewy body disease (DLBD) and Alzheimer's disease with Lewy body pathology. Our study did not directly in-vestigate C-terminal truncated αSyn forms as seeds but measured different aggregation propensities by the RT-QuIC assay in different brain regions and extracts from PD and DLB. Those findings emphasize the need to determine if those differences are related to the amounts and types of C-terminal truncated αSyn forms in different αSynucleinopathies.

One limitation of using the RT-QuIC and the PMCA assays to measure αSyn aggregation propensity in biological samples is the inability to characterize the actual strains and seeds in samples. In our study, using different substrates, we reported heterogeneity in the αSyn aggregation propensity between different brain regions and extracts from PD and DLB patients. However, it remains unclear whether those differences are driven by specific endogenous differences in samples, which include different nature (forms) and amounts of the αSyn strains and seeds. The actual strains and seeds that cause the propagation of αSyn aggregation is of a great interest in the field of αSynucleinopathies. Quantification of different forms of αSyn—full length versus truncated, phosphorylated versus non-phosphorylated and monomeric versus oligomeric versus fibrils—may allow testing correlations between different αSyn forms in biological samples and in vitro aggregation propensity. Importantly, specific depletion of different αSyn forms in the sample is required to determine which form(s) cause the aggregation in the RT-QuIC assays but such studies cannot be done at present due to the lack of tools like antibodies specific to the different αSyn forms. Nonetheless, it should be noted that one advantage of the RT-QuIC technique is its strain/seed-agnostic nature which allows the comparison of seeding propensity across biological samples in a semi-quantitative manner as deployed in our study.

In conclusion, our study demonstrated the value of using C-terminally truncated αSyn 1-130 and αSyn 1-115 as substrates for RT-QuIC assays to differentiate PD and DLB from healthy controls and differentiating PD from DLB. Biochemical characterization of the end products from RT-QuIC, particularly with the αSyn 1-130 protein, suggests differences in PK resistance and αSyn aggregate size and fibril length depending on the brain regions and fractions used. Further studies are warranted to investigate αSyn 1-130, and C-terminal truncated αSyn other than 1-115, to differentiate synculeinopathies by their seeding propensity using RT-QuIC.

Different strategies have been used to improve the sensitivity and the specificity of the αSyn RT-QuIC assay for the clinical diagnosis of synucleinopathies using CSF as seed. However, one of the major drawbacks remains the duration of the reaction. A single RT-QuIC experiment using full-length α-Syn takes around five days to reach completion, which is not ideal in a clinical setting. In our study, we have observed a trend towards faster kinetics of aggregation with increasing truncation of α-Syn using brain homogenates fractions as seed. The use of truncated αSyn forms as substrates should be exploited to improve the diagnostic utility of the RT-QuIC assay.

### Example 2—Diagnosis and Treatment of PD

Frontal cortex samples from a 50 year old male were tested to determine the presence of PD or DLB. As described supra, a RT-QuIC assay was performed to compare the seeding efficiency of αSyn 1-130. The test results showed a higher seeding activity as compared to DLB control samples, thus indicating the presence of PD. Appropriate treatment was then pursued.

### Example 3—Diagnosis and Treatment of DLB

Frontal cortex samples from a 60 year old female were tested to determine the presence of PD or DLB. As described supra, a RT-QuIC assay was performed to compare the seeding efficiency of αSyn 1-130. The test results showed a lower seeding activity as compared to PD control samples, thus indicating the presence of DLB. Appropriate treatment was then pursued.

### Example 4—Diagnosis and Treatment of PD

Frontal cortex samples from a 62 year old male were tested to determine the presence of PD or DLB. As described supra, a RT-QuIC assay was performed to compare the seeding efficiency of αSyn 1-130. The test results showed a higher seeding activity as compared to DLB control samples, thus indicating the presence of PD. Appropriate treatment was then pursued.

### Example 5—Diagnosis and Treatment of DLB

Frontal cortex samples from a 70 year old female were tested to determine the presence of PD or DLB. As described supra, a RT-QuIC assay was performed to compare the seeding efficiency of αSyn 1-130. The test results showed a lower seeding activity as compared to PD control samples, thus indicating the presence of DLB. Appropriate treatment was then pursued.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the invention so claimed are inherently or expressly described and enabled herein.

Disclosed embodiments comprise:

Embodiment 1—A method for diagnosing a synucleinopathy comprising:
homogenizing a tissue sample from a patient;
centrifuging the homogenized tissue to form a supernatant and a pellet, wherein said supernatant comprises an aqueous-soluble fraction;
resuspending the pellet in a detergent, then centrifuging the resuspended pellet to form a supernatant comprising a detergent-soluble fraction;
quantifying the seeding activity of the aqueous-soluble and detergent-soluble fractions; and
comparing the differences in seeding activity of the aqueous-soluble and detergent-soluble fractions, wherein said differences are indicative of PD or DLB.

Embodiment 2—the method of embodiment 1, wherein said purified recombinant αSyn comprises at least one of αSyn 1-140, αSyn 1-130, and αSyn 1-115.

Embodiment 3—the method of embodiment 2, wherein said quantifying the seeding activity of the aqueous-soluble and detergent-soluble fractions comprises performing a RT-QuIC assay.

Embodiment 4—the method of embodiment 3, wherein said recombinant αSyn comprises αSyn 1-130 and said tissue sample comprises a frontal cortex sample, and said detergent soluble fraction demonstrates a higher seeding activity in PD samples as compared to DLB samples.

Embodiment 5—the method of embodiment 3, wherein said recombinant αSyn comprises αSyn 1-115 and said tissue sample comprises a frontal cortex sample, and wherein the rate of said seeding activity is higher in said aqueous soluble fraction as compared to the detergent soluble fraction in DLB samples.

Embodiment 6—the method of embodiment 2, wherein said purified recombinant αSyn comprises αSyn 1-115.

Embodiment 7—the method of embodiment 2, wherein said purified recombinant αSyn comprises αSyn 1-130.

Embodiment 8—the method of embodiment 2, wherein said purified recombinant αSyn comprises αSyn 1-140.

Embodiment 9—the method of embodiment 2, further comprising treating a diagnosed synucleinopathy, the treatment comprising administration of at least one of levodopa, zonisamide, valproic acid, quetiapine, pimavanserin, αSyn targeting agents including NPT200-11 and ambroxol, melatonin, and clonazepam.

Embodiment 10—the method of embodiment 9, wherein said synucleinopathy comprises PD.

Embodiment 11—the method of embodiment 9, wherein said synucleinopathy comprises DLB.

Furthermore, numerous references have been made to patents and printed publications throughout this specification. Each of the above-cited references and printed publications are individually incorporated herein by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

1. A method for diagnosing a synucleinopathy comprising:
homogenizing a tissue sample from a patient;
centrifuging the homogenized tissue to form a supernatant and a pellet, wherein said supernatant comprises an aqueous-soluble fraction;
resuspending the pellet in a detergent, then centrifuging the resuspended pellet to form a supernatant comprising a detergent-soluble fraction;
quantifying the seeding activity of the aqueous-soluble and detergent-soluble fractions; and
comparing the differences in seeding activity of the aqueous-soluble and detergent-soluble fractions, wherein said differences are indicative of Parkinson's Disease (PD) or Dementia with Lewy bodies (DLB).

2. The method of claim 3, wherein said purified recombinant αSyn comprises at least one of αSyn 1-140, αSyn 1-130, and αSyn 1-115.

3. The method of claim 1, wherein said quantifying the seeding activity of the aqueous-soluble and detergent-soluble fractions comprises performing a RT-QuIC assay using purified recombinant αSyn.

4. The method of claim 3, wherein said purified recombinant αSyn comprises αSyn 1-130 and said tissue sample comprises a frontal cortex sample, and said detergent soluble fraction demonstrates a higher seeding activity in PD samples as compared to DLB samples.

5. The method of claim 3, wherein said purified recombinant αSyn comprises αSyn 1-115 and said tissue sample comprises a frontal cortex sample, and wherein the rate of said seeding activity is higher in said aqueous soluble fraction as compared to the detergent soluble fraction in DLB samples.

6. The method of claim 2, wherein said purified recombinant αSyn comprises αSyn 1-115.

7. The method of claim 2, wherein said purified recombinant αSyn comprises αSyn 1-130.

8. The method of claim 2, wherein said purified recombinant αSyn comprises αSyn 1-140.

9. The method of claim 2, further comprising treating a diagnosed synucleinopathy, said treatment comprising administration of at least one of levodopa, zonisamide, valproic acid, quetiapine, pimavanserin, αSyn targeting agents, NPT200-11, ambroxol, melatonin, and clonazepam.

10. The method of claim 9, wherein said synucleinopathy comprises PD.

11. The method of claim 9, wherein said synucleinopathy comprises DLB.

12. The method of claim 2, further comprising treating a diagnosed synucleinopathy, said treatment comprising administration of an anti-inflammatory or antioxidant.

13. A method of treating a synucleinopathy in a patient comprising:
quantifying seeding activity of aqueous-soluble and detergent-soluble fractions in a homogenized tissue sample from the patient; and
comparing the differences in seeding activity of the aqueous-soluble and detergent-soluble fractions, wherein said differences are indicative of PD or DLB; and
treating a diagnosed synucleinopathy by administering at least one of levodopa, zonisamide, valproic acid, quetiapine, pimavanserin, αSyn targeting agents, NPT200-11, ambroxol, melatonin, and clonazepam to the patient.

14. The method of claim 13, wherein said quantifying the seeding activity of the aqueous-soluble and detergent-soluble fractions comprises performing a RT-QuIC assay using purified recombinant αSyn.

15. The method of claim 14, wherein said purified recombinant αSyn comprises αSyn 1-130 and said tissue sample comprises a frontal cortex sample, and said detergent soluble fraction demonstrates a higher seeding activity in PD samples as compared to DLB samples.

16. The method of claim 14, wherein said purified recombinant αSyn comprises αSyn 1-115 and said tissue sample comprises a frontal cortex sample, and wherein the rate of said seeding activity is higher in said aqueous soluble fraction as compared to the detergent soluble fraction in DLB samples.

17. The method of claim 14, wherein said purified recombinant αSyn comprises αSyn 1-115.

18. The method of claim 14, wherein said purified recombinant αSyn comprises αSyn 1-130.

19. The method of claim 14, wherein said purified recombinant αSyn comprises αSyn 1-140.

20. A method for diagnosing a synucleinopathy comprising:
comparing the differences in seeding activity of aqueous-soluble and detergent-soluble fractions of a homogenized tissue sample, wherein said differences are indicative of PD or DLB.
